(19) United States
(12) Patent Application Publication
(10) Pub. No.: US 2023/0092769 A1
(43) Pub. Date: Mar. 23, 2023

(54) COMBINATIONAL THERAPY COMPRISING GLP-1 AND/OR GLP-1 ANALOGS, AND INSULIN AND/OR INSULIN ANALOGS

(71) Applicant: SHANGHAI BENEMAE PHARMACEUTICAL CORPORATION, Shanghai (CN)

(72) Inventors: Yajun ZUO, Shanghai (CN); Xiankang FANG, Shanghai (CN); Zhiqiang DU, Shanghai (CN); Jing XIA, Shanghai (CN)

(73) Assignee: SHANGHAI BENEMAE PHARMACEUTICAL CORPORATION, Shanghai (CN)

(21) Appl. No.: 17/813,284

(22) Filed: Jul. 18, 2022

Related U.S. Application Data
(63) Continuation of application No. PCT/CN2021/072268, filed on Jan. 15, 2021.
Application No. PCT/CN2021/072268 is a continuation of application No. PCT/CN2020/072544, filed on Jan. 16, 2020.

(51) Int. Cl. A61K 38/26; A61K 38/28; A61P 3/10

(52) U.S. Cl. CPC A61K 38/26; A61K 38/28; A61P 3/10

(57) ABSTRACT

Disclosed herein are combinational therapies for diabetes comprising a first effective amount of one or more first active ingredients selected from the group consisting of GLP-1 and GLP-1 analogs and a second effective amount of one or more second active ingredients selected from the group consisting of insulin and insulin analogs.

### PRIORITY CLAIM

This application is a continuation of International Patent Application No. PCT/CN2021/072268, filed Jan. 15, 2021, which is a continuation of International Patent Application No. PCT/CN2020/072544, filed Jan. 16, 2020, both of which are incorporated by reference herein in their entirety.

### BACKGROUND

GLP-1 is an insulinotropic peptide acting on GLP-1 receptor expressed particularly on pancreatic insulin-secreting β cells and on neurons of the brain. The native form of GLP-1 is secreted by intestinal L-cells after a meal, and is a strong peptide stimulator of insulin secretion. GLP-1 is a potential therapy for type 2 diabetes. Hoist, Physiol. Rev. 87: 1409-1439 (2007). GLP-1 has two active forms, GLP-1 (7-36) with a C-terminal amide-NH2 and GLP-1 (7-37) with a C-terminal free carboxyl group. Upon entry into circulation, GLP-1 is rapidly degraded by dipeptidyl peptidase-4 (DPP4), resulting in a short half-life of about 2 minutes. Kieffer et al., Endocrinology 136: 3585-3596 (1995). Besides its effect on controlling blood sugar, GLP-1 and its analogs were found to have effects on inducing body weight loss, slowing stomach emptying, and increasing satiety. Monami et al., Exp. Diabetes Res. 2012: 672658 (2012). FDA approved Saxenda (liraglutide 3 mg) in December 2014, which is the first NDA application for GLP-1 analogs, and which is a long-term weight management supplemented with low-calorie diet and increase of physical activity for obesity adults having at least one disease or condition associated with overweight, such as type 2 diabetes. Continuing efforts are made to further improve the efficacy of GLP-1 analogs. This disclosure provides a combinational therapy that further improves the therapeutic effects of GLP-1 analogs.

### SUMMARY

In one aspect, provided is a method of treating a condition associated with elevated blood glucose in a subject. The method entails administering to a subject a first effective amount of one or more first active ingredients selected from the group consisting of GLP-1 and GLP-1 analogs and a second effective amount of one or more second active ingredients selected from the group consisting of insulin and insulin analogs. In some embodiments, the subject has diabetes such as type 1 and type 2 diabetes or has an elevated risk of having elevated blood glucose and/or diabetes such as type 1 and type 2 diabetes. In some embodiments, the GLP-1 analogs are selected from the group consisting of GLP-1 (7-37), GLP-1 (7-36), and GLP-1 (7-35). In some embodiments, the GLP-1 analog is GLP-1 (7-36) (e.g., beinaglutide). In some embodiments, the insulin analogs are selected from the group consisting of rapid-acting insulins (e.g., insulin lispro, insulin aspart, insulin glulisine) and long-acting insulins (e.g., insulin glargine, insulin degludec, and insulin detemir). In some embodiments, the second effective amount (U) of insulin and/or insulin analog(s) and the first effective amount (mg) of GLP-1 and/or GLP-1 analog(s) may be administered in a ratio of between about 10:1 and about 800:1, about 30:1 to about 500:1, about 50:1 to about 250:1, about 70:1 to about 220:1, or about 100:1 to about 200:1, for example, about 10:1, about 20:1, about 30:1, about 40:1, about 50:1, about 60:1, about 60:1, about 70:1, about 80:1, about 90:1, about 100:1, about 150:1, about 200:1, about 250:1, about 300:1, about 350:1, about 400:1, about 450:1, about 500:1, about 550:1, about 600:1, about 650:1, about 700:1, about 750:1 or about 800:1. In some embodiments, the GLP-1 and/or GLP-1 analog(s) (e.g., GLP-1 (7-36) such as beinaglutide) may be administered to a subject (e.g., human) in a range of about 0.00070 mg/kg to about 0.0197 mg/kg body weight, about 0.00071 mg/kg to about 0.0178 mg/kg body weight, about 0.00071 mg/kg to about 0.0159 mg/kg body weight, about 0.00072 mg/kg mg/kg to about 0.014 mg/kg body weight, about 0.00072 mg/kg to about 0.0121 mg/kg body weight, about 0.00072 mg/kg to about 0.01115 mg/kg body weight, about 0.00073 mg/kg to about 0.0102 mg/kg body weight, about 0.00076 mg/kg to about 0.00925 mg/kg body weight, about 0.00080 mg/kg to about 0.0083 mg/kg body weight, about 0.00089 mg/kg to about 0.00792 mg/kg body weight, about 0.00108 mg/kg to about 0.00735 mg/kg body weight, about 0.00127 mg/kg to about 0.00678 mg/kg body weight, about 0.00165 mg/kg to about 0.0064 mg/kg body weight, about 0.00184 mg/kg to about 0.00602 mg/kg body weight, about 0.00203 mg/kg to about 0.00564 mg/kg body weight, about 0.00222 mg/kg to about 0.00545 mg/kg body weight, about 0.0026 mg/kg to about 0.00583 mg/kg body weight, about 0.00279 mg/kg to about 0.00564 mg/kg body weight, or about 0.00298 mg/kg to about 0.00526 mg/kg body weight. In some embodiments, the GLP-1 and/or GLP-1 analog(s) (e.g., GLP-1 (7-36) such as beinaglutide) may be administered in a range of about 0.001 mg/kg to about 10.0 mg/kg body weight, about 0.003 mg/kg to about 9.0 mg/kg body weight, about 0.005 mg/kg to about 8.0 mg/kg body weight, about 0.01 mg/kg to about 7.0 mg/kg body weight, about 0.01 mg/kg to about 6.0 mg/kg body weight, about 0.01 mg/kg to about 5.5 mg/kg body weight, about 0.015 mg/kg to about 5.0 mg/kg body weight, about 0.03 mg/kg to about 4.5 mg/kg body weight, about 0.05 mg/kg to about 4.0 mg/kg body weight, about 0.1 mg/kg to about 3.8 mg/kg body weight, about 0.2 mg/kg to about 3.5 mg/kg body weight, about 0.3 mg/kg to about 3.2 mg/kg body weight, about 0.5 mg/kg to about 3.0 mg/kg body weight, about 0.6 mg/kg to about 2.8 mg/kg body weight, about 0.7 mg/kg to about 2.6 mg/kg body weight, about 0.8 mg/kg to about 2.5 mg/kg body weight, about 1.0 mg/kg to about 2.7 mg/kg body weight, about 1.1 mg/kg to about 2.6 mg/kg body weight, or about 1.2 mg/kg to about 2.4 mg/kg body weight. In some embodiments, the GLP-1 and/or GLP-1 analog(s) (e.g., GLP-1 (7-36) such as beinaglutide) may be administered at a daily dosage of about 0.01 mg/d to about 1 mg/d, about 0.06 mg/d to 0.6 mg/d, or about 0.12 mg/d to about 0.4 mg/d.

In some embodiments, the GLP-1 and/or GLP-1 analog(s) (e.g., GLP-1 (7-36) such as beinaglutide) may be administered once a day, twice a day, three times a day, or four times a day. In some embodiments, the insulin and/or insulin analog(s) (e.g., insulin glargine) may be administered to a subject (e.g., human) in a range from about 2 U/d to about 100 U/d, about 6 U/d to about 60 U/d, about 12 U/d to about 40 U/d, about 20 U/d to about 30 U/d, or about 15 U/d.

In some embodiments, the first effective amount of the GLP-1 and/or GLP-1 analog(s) (e.g., GLP-1 (7-36) such as beinaglutide) and/or the second effective amount of the insulin and/or insulin analog(s) (e.g., insulin glargine) are administered once a day, twice a day, three times a day, or four times a day. In some embodiments, the GLP-1 and/or GLP-1 analog(s) (e.g., GLP-1 (7-36) such as beinaglutide) may be administered before, during, or after the administration of the insulin and/or insulin analog(s) (e.g., insulin glargine).

In another aspect, provided is a combinational therapy comprising a first effective amount of the GLP-1 and/or GLP-1 analog(s) (e.g., GLP-1 (7-36) such as beinaglutide) and an effective amount of the insulin and/or insulin analog(s) (e.g., insulin glargine). The combinational therapy is for treating a condition associated with elevated blood glucose in a subject. In some embodiments, the subject has diabetes such as type 1 and type 2 diabetes or has an elevated risk of having elevated blood glucose and/or diabetes such as type 1 and type 2 diabetes. In some embodiments, the GLP-1 analogs are selected from the group consisting of GLP-1 (7-37), GLP-1 (7-36), and GLP-1 (7-35). In some embodiments, the GLP-1 analog is GLP-1 (7-36) (e.g., beinaglutide). In some embodiments, the insulin analogs are selected from the group consisting of rapid-acting insulins (e.g., insulin lispro, insulin aspart, insulin glulisine) and long-acting insulin (e.g., insulin glargine, insulin degludec, and insulin detemir). In some embodiments, the second effective amount (U) of the insulin and/or insulin analog(s) and the first effective amount (mg) of the GLP-1 and/or GLP-1 analog(s) are administered in a ratio of between about 10:1 and about 800:1, about 30:1 to about 500:1m about 50:1 to about 250:1, about 70:1 to about 220:1, or about 100:1 to about 200:1, for example, about 10:1, about 20:1, about 30:1, about 40:1, about 50:1, about 60:1, about 60:1, about 70:1, about 80:1, about 90:1, about 100:1, about 150:1, about 200:1, about 250:1, about 300:1, about 350:1, about 400:1, about 450:1, about 500:1, about 550:1, about 600:1, about 650:1, about 700:1, about 750:1 or about 800:1. In some embodiments, the GLP-1 and/or GLP-1 analog(s) (e.g., GLP-1 (7-36) such as beinaglutide) may be administered to a subject (e.g., human) in a range of about 0.00070 mg/kg to about 0.0197 mg/kg body weight, about 0.00071 mg/kg to about 0.0178 mg/kg body weight, about 0.00071 mg/kg to about 0.0159 mg/kg body weight, about 0.00072 mg/kg mg/kg to about 0.014 mg/kg body weight, about 0.00072 mg/kg to about 0.0121 mg/kg body weight, about 0.00072 mg/kg to about 0.01115 mg/kg body weight, about 0.00073 mg/kg to about 0.0102 mg/kg body weight, about 0.00076 mg/kg to about 0.00925 mg/kg body weight, about 0.00080 mg/kg to about 0.0083 mg/kg body weight, about 0.00089 mg/kg to about 0.00792 mg/kg body weight, about 0.00108 mg/kg to about 0.00735 mg/kg body weight, about 0.00127 mg/kg to about 0.00678 mg/kg body weight, about 0.00165 mg/kg to about 0.0064 mg/kg body weight, about 0.00184 mg/kg to about 0.00602 mg/kg body weight, about 0.00203 mg/kg to about 0.00564 mg/kg body weight, about 0.00222 mg/kg to about 0.00545 mg/kg body weight, about 0.0026 mg/kg to about 0.00583 mg/kg body weight, about 0.00279 mg/kg to about 0.00564 mg/kg body weight, or about 0.00298 mg/kg to about 0.00526 mg/kg body weight. In some embodiments, the GLP-1 and/or GLP-1 analog(s) (e.g., GLP-1 (7-36) such as beinaglutide) may be administered in a range of about 0.001 mg/kg to about 10.0 mg/kg body weight, about 0.003 mg/kg to about 9.0 mg/kg body weight, about 0.005 mg/kg to about 8.0 mg/kg body weight, about 0.01 mg/kg to about 7.0 mg/kg body weight, about 0.01 mg/kg to about 6.0 mg/kg body weight, about 0.01 mg/kg to about 5.5 mg/kg body weight, about 0.015 mg/kg to about 5.0 mg/kg body weight, about 0.03 mg/kg to about 4.5 mg/kg body weight, about 0.05 mg/kg to about 4.0 mg/kg body weight, about 0.1 mg/kg to about 3.8 mg/kg body weight, about 0.2 mg/kg to about 3.5 mg/kg body weight, about 0.3 mg/kg to about 3.2 mg/kg body weight, about 0.5 mg/kg to about 3.0 mg/kg body weight, about 0.6 mg/kg to about 2.8 mg/kg body weight, about 0.7 mg/kg to about 2.6 mg/kg body weight, about 0.8 mg/kg to about 2.5 mg/kg body weight, about 1.0 mg/kg to about 2.7 mg/kg body weight, about 1.1 mg/kg to about 2.6 mg/kg body weight, or about 1.2 mg/kg to about 2.4 mg/kg body weight. In some embodiments, the insulin and/or insulin analog(s) (e.g., insulin glargine) may be administered to a subject (e.g., human) in a range from about 2 U/d to about 100 U/d, about 6 U/d to about 60 U/d, about 12 U/d to about 40 U/d, about 20 U/d to about 30 U/d, or about 15 U/d. In some embodiments, the GLP-1 and/or GLP-1 analog(s) (e.g., GLP-1 (7-36) such as beinaglutide) may be administered at a daily dosage of about 0.01 mg/d to about 1 mg/d, about 0.06 mg/d to 0.6 mg/d, or about 0.12 mg/d to about 0.4 mg/d.

In some embodiments, the first effective amount of GLP-1 and/or GLP-1 analogs (e.g., GLP-1 (7-36) such as beinaglutide) and the second effective amount of the insulin and/or insulin analogs (e.g., insulin glargine) are formulated in a single composition. In some embodiments, the first effective amount of the GLP-1 and/or GLP-1 analogs (e.g., GLP-1 (7-36) such as beinaglutide) and the second effective amount of the insulin and/or insulin analogs (e.g., insulin glargine) are formulated in two separate compositions and combined during treatment.

In yet another aspect, provided is a kit comprising a first effective amount of the GLP-1 and/or GLP-1 analog(s) (e.g., GLP-1 (7-36) such as beinaglutide) and a second effective amount of the insulin and/or insulin analog(s) (e.g., insulin glargine). The kit is used for treating a condition associated with elevated blood glucose in a subject. In some embodiments, the subject has diabetes such as type 1 and type 2 diabetes or has an elevated risk of having elevated blood glucose and/or diabetes such as type 1 and type 2 diabetes. In some embodiments, 1) the GLP-1 and/or GLP-1 analog(s) (e.g., GLP-1 (7-36) such as beinaglutide) and 2) the insulin and/or insulin analog(s) (e.g., insulin glargine) are formulated in a single composition. In some embodiments, 1) the GLP-1 and/or GLP-1 analog(s) (e.g., GLP-1 (7-36) such as beinaglutide) and 2) the insulin and/or insulin analog(s) (e.g., insulin glargine) are formulated in two separate compositions. In some embodiments, the kit further comprises instructions for using the same.

### BRIEF DESCRIPTION OF THE DRAWINGS

FIG. 1 shows the blood glucose levels of tested db/db mice (n=5) at various doses of beinaglutide (Bei) from 0.2 mg/kg to 2 mg/kg in combination of 60 U/kg of insulin glargine (IGla) over a period of 4 hours after beinaglutide injection.

FIG. 2 shows the blood glucose levels of tested db/db mice (n=5) at various doses of beinaglutide (Bei) from 0.1 mg/kg to 1.2 mg/kg in combination of 60 U/kg of insulin glargine (IGla) over a period of 4 hours after beinaglutide injection.

FIG. 3 shows the blood glucose levels of tested STZ-induced diabetic mice (n=6) after a single injection of beinaglutide (Bei), insulin glargine (IGla), or a combination of Bei and IGla. The doses were 0.1 mg/kg and 2.4 mg/kg for Bei, and 5 U/kg and 10 U/kg for IGla, respectively. The doses for the combo groups were 0.05 mg/kg of Bei and 5 U/kg of IGla, and 0.1 mg/kg of Bei and 10 U/kg of IGla, respectively.

FIG. 4 shows the area under curve (AUC) of blood glucose levels of tested STZ-induced diabetic mice (n=6) after a single injection of beinaglutide (Bei), insulin glargine (IGla), or a combination of Bei and IGla. The doses were 0.1 mg/kg and 2.4 mg/kg for Bei, and 5 U/kg and 10 U/kg for IGla, respectively. The doses for the combo groups were 0.05 mg/kg of Bei and 5 U/kg of IGla, and 0.1 mg/kg of Bei and 10 U/kg of IGla, respectively.

FIG. 5 shows the blood glucose levels of individual mouse for each group (A-G) after a single injection of beinaglutide (Bei), insulin glargine (IGla), or a combination of Bei and IGla. The doses were 0.1 mg/kg and 2.4 mg/kg for Bei, and 5 U/kg and 10 U/kg for IGla, respectively. The doses for the combo groups were 0.05 mg/kg of Bei and 5 U/kg of IGla, and 0.1 mg/kg of Bei and 10 U/kg of IGla, respectively.

FIG. 6 shows the body weight change of tested STZ-induced diabetic mice (n=6) after multiple injections of beinaglutide (Bei, 0.1 mg/kg), insulin glargine (IGla, 5 U/kg), or a combination of Bei and IGla (0.05 mg/kg of Bei, 5 U/kg of IGla, respectively) for four weeks. The mice were treated twice per day for 28 days for each group. The body weight change was calculated by comparing the initial body weight of Day 1.

FIG. 7 shows the body weight change on day 28 comparing to the initial body weight on day 1. The mice were treated twice per day for 28 days. The dose was 0.1 mg/kg for beinaglutide (Bei), 5 U/kg for insulin glargine (IGla), and 0.05 mg/kg of Bei and 5 U/kg of IGla for the combo group, respectively.

FIG. 8 shows the fasting blood glucose of tested STZ-induced diabetic mice (n=6) on Day 28 after 6 hours of fasting.

FIG. 9 shows the blood glucose levels of oral glucose tolerance test (OGTT) of tested STZ-induced diabetic mice (n=6) on Day 28. The mice fasted for about 6 hours were orally administrated with glucose solution (1 g/kg). Blood glucose was measured at indicated time points.

FIG. 10 shows the area under curve (AUC) of blood glucose levels over 120 minutes during OGTT.

### DETAILED DESCRIPTION

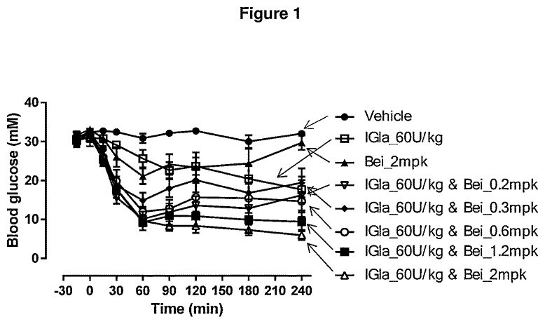

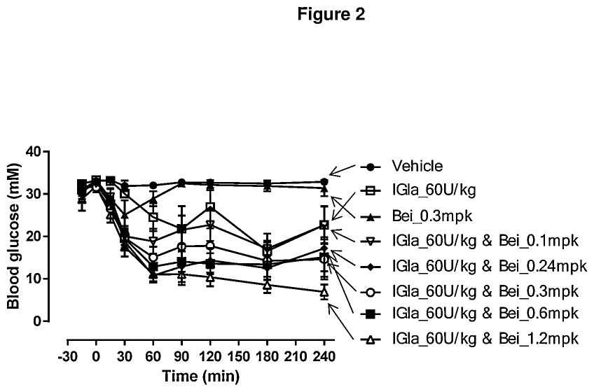

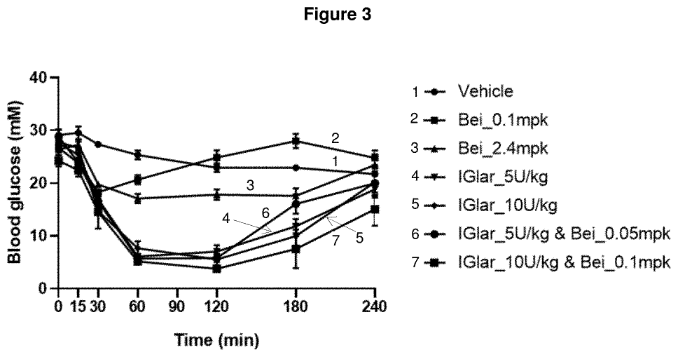

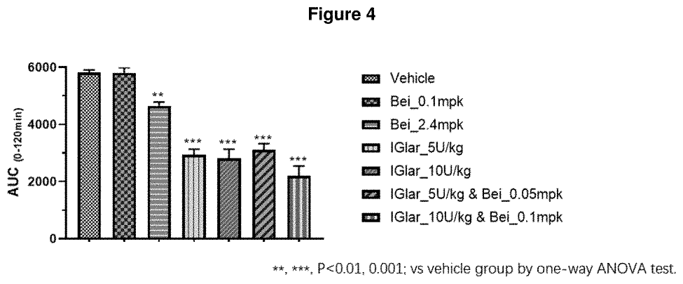

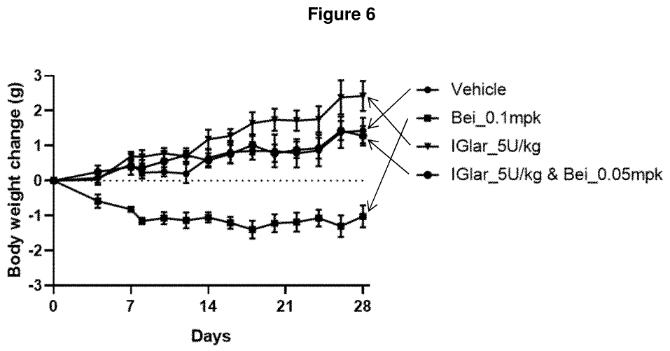

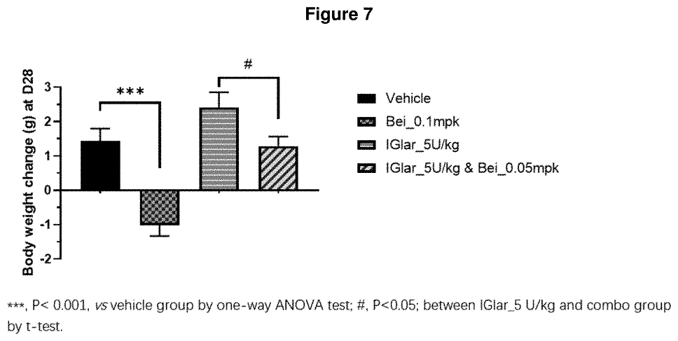

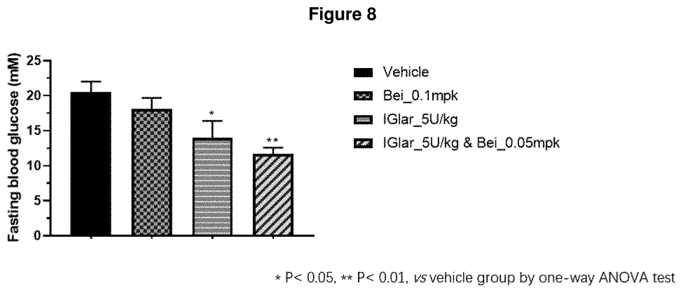

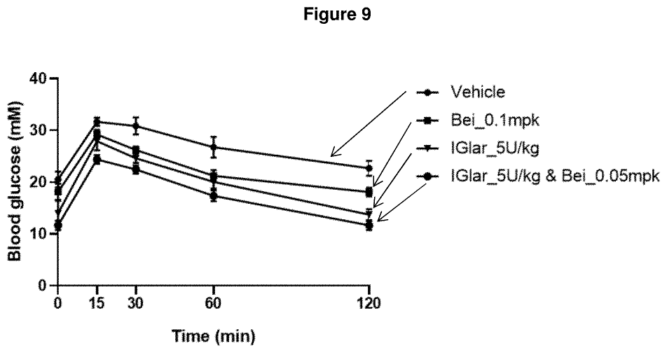

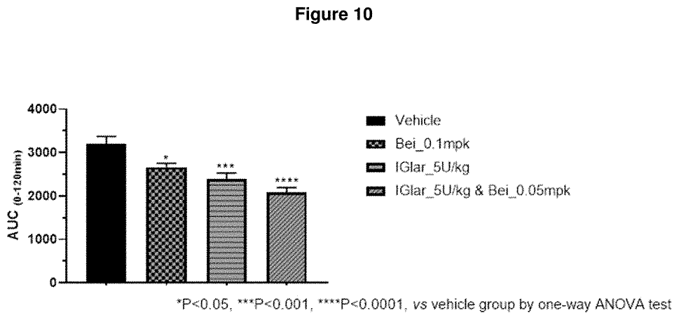

It is an important goal of diabetic treatments to control blood glucose level to an optimal range. Blood glucose levels which are significantly above (hyperglycemia) or below (hypoglycemia) the optimal range can be problematic and dangerous. Diabetic patients, especially those treated with insulin, are at risk for developing hypoglycemia. As shown in Example 1 disclosed herein, none of the subjects treated with various embodiments of combinational therapy of beinaglutide and insulin glargine developed hypoglycemia. Furthermore, Example 1 showed a beinaglutide dose-dependent synergistic improvement of blood glucose control in subjects treated with a constant dose of insulin glargine (60 U/kg), see FIG. 1, beinaglutide administered at 0.2 mg/kg, 0.3 mg/kg, 0.6 mg/kg, 1.2 mg/kg, and 2 mg/kg; and FIG. 2, beinaglutide administered at 0.1 mg/kg, 0.24 mg/kg, 0.3 mg/kg, 0.6 mg/kg, and 1.2 mg/kg. Example 2 showed that the efficacy of the combinational therapy of beinaglutide and insulin glargine in glycemic control was more stable, durable, and faster compared to insulin glargine administration alone (FIGS. 3 and 4); although insulin glargine administration alone induced weight gain significantly higher than the combinational therapy of beinaglutide and insulin glargine (FIGS. 6 and 7); the combinational therapy of beinaglutide and insulin glargine significantly reduced fasting blood glucose compared to vehicle group (FIG. 8); and the combinational therapy of beinaglutide and insulin glargine had synergistic effect in lowering the blood glucose level compared to beinaglutide alone and insulin glargine alone (FIGS. 9 and 10). It is contemplated that a combinational therapy of 1) the GLP-1 and/or GLP-1 analog(s) and 2) the insulin and/or insulin analog(s) disclosed herein may achieve desired therapeutic effects in subjects in need of blood glucose control, e.g., diabetic subjects.

Disclosed herein is a combinational therapy comprising a first effective amount of one or more first active ingredients selected from the group consisting of GLP-1 and GLP-1 analogs (e.g., GLP-1 (7-36) such as beinaglutide) and a second effective amount of one or more first active ingredients selected from the group consisting of insulin and insulin analogs (e.g., insulin glargine), wherein one or more first active ingredients and the one or more second active ingredients may be combined at different doses and/or ratios. The combinational therapy can be used for treating various conditions associated with an elevated blood glucose level such as diabetes including type 1 and type 2 diabetes. The combinational therapy improves the therapeutic effects comparing to monotherapy of 1) the GLP-1 and/or GLP-1 analog(s) or 2) the insulin and/or insulin analog(s). For example, as provided in Example 1 disclosed herein, an embodiment of the combinational therapy of beinaglutide and insulin glargine improved blood glucose control in subjects compared to subjects treated with insulin glargine or beinaglutide alone.

In the context of this disclosure, the phrase a “therapeutically effective amount” or an “effective amount” of a pharmaceutical composition comprising 1) the GLP-1 (and/or GLP-1 analogs (e.g., GLP-1 (7-36) such as beinaglutide)) and/or 2) the insulin and/or insulin analogs (e.g., insulin glargine) as used herein is an amount of the pharmaceutical composition that produces a desired therapeutic effect in a subject, such as treating diabetes. In certain embodiments, the therapeutically effective amount is an amount of the pharmaceutical composition that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. In some embodiments, a therapeutically effective amount is the minimal amount that produces a therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological art will be able to determine a therapeutically effective amount through routine experimentation, namely by monitoring a subject's response to administration of the pharmaceutical composition and adjusting the dosage accordingly. For additional guidance, see, e.g., Remington: The Science and Practice of Pharmacy, 22nd Edition, Pharmaceutical Press, London, 2012, and Goodman & Gilman's The Pharmacological Basis of Therapeutics, 12th Edition, McGraw-Hill, New York, N.Y., 2011, the entire disclosures of which are incorporated by reference herein.

The terms “treat,” “treating,” and “treatment” as used herein with regard to a condition refers to alleviating the condition partially or entirely, decreasing the likelihood of occurrence or recurrence of the condition, slowing the progression or development of the condition, or eliminating, reducing, or slowing the development of one or more symptoms associated with the condition.

As used herein, the term “subject” refers to a mammalian subject, preferably human. In certain embodiments, the subject has diabetes such as type 1 and type 2 diabetes or has an elevated risk of having elevated blood glucose and/or diabetes such as type 1 and type 2 diabetes. The phrases “subject” and “patient” can be used interchangeably herein.

### Combinational Therapy and Uses Thereof

In certain embodiments, the composition disclosed herein comprises either or both of 1) the GLP-1 and/or GLP-1 analog(s) and 2) the insulin and/or insulin analog(s).

In some embodiments, the GLP-1 analog is selected from the group consisting of GLP-1 (7-37), GLP-1 (7-36), and GLP-1 (7-35). Unless specified otherwise, GLP-1 and a GLP-1 analog may have a C-terminal free carboxyl group or a C-terminal amide group. For example, the GLP-1 analog can be a recombinant human GLP-1 (7-36) peptide having the sequence of: His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 1), which may be referred to as beinaglutide in this disclosure. Beinaglutide has a molecular formula of C₁₄₉H₂₂₅N₃₉O₄₆, and a molecular weight of 3,298.7. Beinaglutide is essentially the same as the active form of circulating GLP-1 except for the endogenous amidation, where NH₂ in the natural form is replaced by OH group in the recombinant peptide. Beinaglutide includes a C-terminal free carboxyl group. In other embodiments, GLP-1 (7-35) or GLP-1 (7-37) can be used in the disclosed technology. The sequences of GLP-1 (7-35) having a C-terminal free carboxyl group and GLP-1 (7-37) having a C-terminal free carboxyl group are as follows:

(SEQ ID NO: 2)
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly,
and
(SEQ ID NO: 3)
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly.

In some embodiments, the GLP-1 and/or GLP-1 analog(s) (e.g., GLP-1 (7-36) such as beinaglutide) may be administered to a subject (e.g., human) in a range of about 0.00070 mg/kg to about 0.0197 mg/kg body weight, about 0.00071 mg/kg to about 0.0178 mg/kg body weight, about 0.00071 mg/kg to about 0.0159 mg/kg body weight, about 0.00072 mg/kg mg/kg to about 0.014 mg/kg body weight, about 0.00072 mg/kg to about 0.0121 mg/kg body weight, about 0.00072 mg/kg to about 0.01115 mg/kg body weight, about 0.00073 mg/kg to about 0.0102 mg/kg body weight, about 0.00076 mg/kg to about 0.00925 mg/kg body weight, about 0.00080 mg/kg to about 0.0083 mg/kg body weight, about 0.00089 mg/kg to about 0.00792 mg/kg body weight, about 0.00108 mg/kg to about 0.00735 mg/kg body weight, about 0.00127 mg/kg to about 0.00678 mg/kg body weight, about 0.00165 mg/kg to about 0.0064 mg/kg body weight, about 0.00184 mg/kg to about 0.00602 mg/kg body weight, about 0.00203 mg/kg to about 0.00564 mg/kg body weight, about 0.00222 mg/kg to about 0.00545 mg/kg body weight, about 0.0026 mg/kg to about 0.00583 mg/kg body weight, about 0.00279 mg/kg to about 0.00564 mg/kg body weight, or about 0.00298 mg/kg to about 0.00526 mg/kg body weight. In some embodiments, the GLP-1 and/or GLP-1 analog(s) (e.g., GLP-1 (7-36) such as beinaglutide) may be administered in a range of about 0.001 mg/kg to about 10.0 mg/kg body weight, about 0.003 mg/kg to about 9.0 mg/kg body weight, about 0.005 mg/kg to about 8.0 mg/kg body weight, about 0.01 mg/kg to about 7.0 mg/kg body weight, about 0.01 mg/kg to about 6.0 mg/kg body weight, about 0.01 mg/kg to about 5.5 mg/kg body weight, about 0.015 mg/kg to about 5.0 mg/kg body weight, about 0.03 mg/kg to about 4.5 mg/kg body weight, about 0.05 mg/kg to about 4.0 mg/kg body weight, about 0.1 mg/kg to about 3.8 mg/kg body weight, about 0.2 mg/kg to about 3.5 mg/kg body weight, about 0.3 mg/kg to about 3.2 mg/kg body weight, about 0.5 mg/kg to about 3.0 mg/kg body weight, about 0.6 mg/kg to about 2.8 mg/kg body weight, about 0.7 mg/kg to about 2.6 mg/kg body weight, about 0.8 mg/kg to about 2.5 mg/kg body weight, about 1.0 mg/kg to about 2.7 mg/kg body weight, about 1.1 mg/kg to about 2.6 mg/kg body weight, or about 1.2 mg/kg to about 2.4 mg/kg body weight. In some embodiments, the GLP-1 and/or GLP-1 analog(s) (e.g., GLP-1 (7-36) such as beinaglutide) may be administered at a daily dosage of about 0.01 mg/d to about 1 mg/d, about 0.06 mg/d to 0.6 mg/d, or about 0.12 mg/d to about 0.4 mg/d. In some embodiments, the GLP-1 and/or GLP-1 analog(s) (e.g., GLP-1 (7-36) such as beinaglutide) may be administered once a day, twice a day, three times a day, or four times a day.

In some embodiments, the insulin analogs are selected from the group consisting of rapid-acting insulins (e.g., insulin lispro, insulin aspart, insulin glulisine) and long-acting insulin (e.g., insulin glargine, insulin degludec, and insulin detemir).

In some embodiments, the second effective amount (U) of the insulin and/or insulin analog(s) and the first effective amount (mg) of the GLP-1 and/or GLP-1 analog(s) are administered in a ratio of between about 10:1 and about 800:1, about 30:1 to about 500:1m about 50:1 to about 250:1, about 70:1 to about 220:1, or about 100:1 to about 200:1, for example, about 10:1, about 20:1, about 30:1, about 40:1, about 50:1, about 60:1, about 60:1, about 70:1, about 80:1, about 90:1, about 100:1, about 150:1, about 200:1, about 250:1, about 300:1, about 350:1, about 400:1, about 450:1, about 500:1, about 550:1, about 600:1, about 650:1, about 700:1, about 750:1 or about 800:1. In some embodiments, the GLP-1 and/or GLP-1 analog(s) (e.g., GLP-1 (7-36) such as beinaglutide) may be administered to a subject (e.g., human) in a range of about 0.00070 mg/kg to about 0.0197 mg/kg body weight, about 0.00071 mg/kg to about 0.0178 mg/kg body weight, about 0.00071 mg/kg to about 0.0159 mg/kg body weight, about 0.00072 mg/kg mg/kg to about 0.014 mg/kg body weight, about 0.00072 mg/kg to about 0.0121 mg/kg body weight, about 0.00072 mg/kg to about 0.01115 mg/kg body weight, about 0.00073 mg/kg to about 0.0102 mg/kg body weight, about 0.00076 mg/kg to about 0.00925 mg/kg body weight, about 0.00080 mg/kg to about 0.0083 mg/kg body weight, about 0.00089 mg/kg to about 0.00792 mg/kg body weight, about 0.00108 mg/kg to about 0.00735 mg/kg body weight, about 0.00127 mg/kg to about 0.00678 mg/kg body weight, about 0.00165 mg/kg to about 0.0064 mg/kg body weight, about 0.00184 mg/kg to about 0.00602 mg/kg body weight, about 0.00203 mg/kg to about 0.00564 mg/kg body weight, about 0.00222 mg/kg to about 0.00545 mg/kg body weight, about 0.0026 mg/kg to about 0.00583 mg/kg body weight, about 0.00279 mg/kg to about 0.00564 mg/kg body weight, or about 0.00298 mg/kg to about 0.00526 mg/kg body weight. In some embodiments, the GLP-1 and/or GLP-1 analog(s) (e.g., GLP-1 (7-36) such as beinaglutide) may be administered in a range of about 0.001 mg/kg to about 10.0 mg/kg body weight, about 0.003 mg/kg to about 9.0 mg/kg body weight, about 0.005 mg/kg to about 8.0 mg/kg body weight, about 0.01 mg/kg to about 7.0 mg/kg body weight, about 0.01 mg/kg to about 6.0 mg/kg body weight, about 0.01 mg/kg to about 5.5 mg/kg body weight, about 0.015 mg/kg to about 5.0 mg/kg body weight, about 0.03 mg/kg to about 4.5 mg/kg body weight, about 0.05 mg/kg to about 4.0 mg/kg body weight, about 0.1 mg/kg to about 3.8 mg/kg body weight, about 0.2 mg/kg to about 3.5 mg/kg body weight, about 0.3 mg/kg to about 3.2 mg/kg body weight, about 0.5 mg/kg to about 3.0 mg/kg body weight, about 0.6 mg/kg to about 2.8 mg/kg body weight, about 0.7 mg/kg to about 2.6 mg/kg body weight, about 0.8 mg/kg to about 2.5 mg/kg body weight, about 1.0 mg/kg to about 2.7 mg/kg body weight, about 1.1 mg/kg to about 2.6 mg/kg body weight, or about 1.2 mg/kg to about 2.4 mg/kg body weight. In some embodiments, the insulin and/or insulin analog(s) (e.g., insulin glargine) may be administered to a subject (e.g., human) in a range from about 2 U/d to about 100 U/d, about 6 U/d to about 60 U/d, about 12 U/d to about 40 U/d, about 20 U/d to about 30 U/d, or about 15 U/d. In some embodiments, the first effective amount of the GLP-1 and/or GLP-1 analog(s) (e.g., GLP-1 (7-36) such as beinaglutide) and/or the second effective amount of the insulin and/or insulin analog(s) (e.g., insulin glargine) may be administered once a day, twice a day, three times a day, or four times a day. In some embodiments, the GLP-1 and/or GLP-1 analog(s) (e.g., GLP-1 (7-36) such as beinaglutide) may be administered before, during, or after the administration of the insulin and/or insulin analog(s) (e.g., insulin glargine). In certain embodiments, the GLP-1 and/or GLP-1 analog(s) (e.g., GLP-1 (7-36) such as beinaglutide) may be administered about 5 min to about 30 min, about 10 min to about 20 min, or about 15 min before or after the administration of the insulin and/or insulin analogs (e.g., insulin glargine). In some embodiments, the GLP-1 and/or GLP-1 analog(s) (e.g., GLP-1 (7-36) such as beinaglutide) may be administered at a daily dosage of about 0.01 mg/d to about 1 mg/d, about 0.06 mg/d to 0.6 mg/d, or about 0.12 mg/d to about 0.4 mg/d.

In some embodiments, a therapeutically effective amount of the insulin and/or insulin analog(s) disclosed herein (e.g., insulin glargine) may be in the range from about 2 U/d to about 100 U/d, about 6 U/d to about 60 U/d, about 12 U/d to about 40 U/d, about 20 U/d to about 30 U/d, or about 15 U/d. In some embodiments, the insulin and/or insulin analogs (e.g., insulin glargine, U) and the GLP-1 and/or GLP-1 analogs (e.g., beinaglutide, mg) are combined at a ratio ranging from 10:1 to 800:1, e.g., 30:1, 50:1, 100:1, 200:1, 250:1, 300:1, or 600:1.

In addition to either or both of 1) the GLP-1 and/or GLP-1 analog(s) (e.g., GLP-1 (7-36) such as beinaglutide) and 2) the insulin and/or insulin analog(s) (e.g., insulin glargine), the pharmaceutical composition disclosed herein may contain one or more pharmaceutically acceptable excipients, and/or buffer (e.g., histidine-hydrochloric acid (histidine-HCl), sodium citrate-citric acid, disodium hydrogen phosphate-citric acid, NaOH-citric acid, sodium acetate-acetic acid (NaAC-HAC), succinate-succinic acid, lactate-lactic acid, glutaminate-glutamic acid, malate-malic acid, benzoate-benzoic acid, tartrate-tartaric acid or glycine-hydrochloric acid (Gly-HCl) or any combinations thereof) salt to maintain the desired pH range of the composition. Additional ingredients for the pharmaceutical composition may include one or more of preservatives (e.g., phenol, benzyl alcohol, methyl p-hydroxybenzoate, ethyl p-hydroxybenzoate, propyl p-hydroxybenzoate, butyl p-hydroxybenzoate, chlorobutanol, 2-phenoxyethanol, 2-phenethyl alcohol, benzalkonium chloride (bromide), merthiolate or any combinations thereof), isotonic agents (polyol, sodium chloride, sugar or any combinations thereof; wherein, the polyol is mannitol, sorbitol, inositol, xylitol, glycerin, propylene glycol or any combinations thereof; and the sugar is sucrose, trehalose, lactose, fructose, glucose or any combinations thereof), and dissolution enhancers (e.g., Tween 20, Tween 40, Tween 80, Span 20, Span 40, Span 80, Poloxamer 188, Pluronic F68, Brij 35, dextran-20, PEG 400, PEG 1000, PEG 1500, PEG 2000, propylene glycol or any combinations thereof).

The pharmaceutical composition is formulated suitable for a particular administration route. For example, the pharmaceutical composition can be injected subcutaneously or intravenously, or be administered by infusion or oral administration. In some embodiments, both of 1) the GLP-1 and/or GLP-1 analog(s) (e.g., GLP-1 (7-36) such as beinaglutide) and 2) the insulin and/or insulin analog(s) (e.g., insulin glargine) are formulated in a single composition for simultaneous administration. In some embodiments, 1) the GLP-1 and/or GLP-1 analog(s) (e.g., GLP-1 (7-36) such as beinaglutide) and 2) the insulin and/or insulin analog(s) (e.g., insulin glargine) are formulated into two separate compositions such that different doses or ratios of 1) the GLP-1 and/or GLP-1 analog(s) and 2) the insulin and/or insulin analog(s) can be combined for simultaneous or sequential administration. For example, the GLP-1 and/or GLP-1 analog(s) (e.g., GLP-1 (7-36) such as beinaglutide) may be administered before, during, or after the administration of the insulin and/or insulin analog(s) (e.g., insulin glargine). In another example, 1) the GLP-1 and/or GLP-1 analog(s) (e.g., GLP-1 (7-36) such as beinaglutide) and 2) the insulin and/or insulin analog(s) (e.g., insulin glargine) may be delivered once a day, twice a day, three times a day or four times day in various combinations. For example, one dose of the insulin and/or insulin analog(s) (e.g., insulin glargine) may be followed by one or more doses of the GLP-1 and/or GLP-1 analog(s) (e.g., GLP-1 (7-36) such as beinaglutide), and vice versa. When multiple doses of the insulin and/or insulin analog(s) (e.g., insulin glargine) or the GLP-1 and/or GLP-1 analog(s) (e.g., GLP-1 (7-36) such as beinaglutide) are administered, it is not necessary that the same dose is administered each time to the subject. It is possible to administer a first dose of 1) the GLP-1 and/or GLP-1 analog(s) (e.g., GLP-1 (7-36) such as beinaglutide) and/or 2) the insulin and/or insulin analog(s) (e.g., insulin glargine), and then adjust the subsequent dose(s) higher or lower, or even skip a dose of either or both of 1) the GLP-1 and/or GLP-1 analog(s) (e.g., GLP-1 (7-36) such as beinaglutide) and 2) the insulin and/or insulin analog(s) (e.g., insulin glargine), depending on the patient's blood glucose level in response to the first dose.

Also disclosed is a kit comprising 1) the GLP-1 and/or GLP-1 analog(s) (e.g., GLP-1 (7-36) such as beinaglutide), 2) the insulin and/or insulin analog(s) (e.g., insulin glargine), and optionally 3) instructions of using the same. The GLP-1 (and/or GLP-1 analog(s) (e.g., GLP-1 (7-36) such as beinaglutide)) and the insulin (and/or insulin analog(s) (e.g., insulin glargine)) may be included in a single composition or in two separate compositions. When the GLP-1 (and/or GLP-1 analog(s) (e.g., GLP-1 (7-36) such as beinaglutide)) and the insulin (and/or insulin analog(s) (e.g., insulin glargine)) are provided in two separate compositions, they can be administered simultaneously or sequentially in various combinations. For example, the instructions will provide which of the GLP-1 (and/or GLP-1 analog(s) (e.g., GLP-1 (7-36) such as beinaglutide)) and the insulin (and/or insulin analog(s) (e.g., insulin glargine)) will be administered first at what dose, how many doses of each will be administered in a day, and as needed if one dose of the insulin (and/or insulin analog(s) (e.g., insulin glargine)) should be followed by one or more doses of the GLP-1 (and/or GLP-1 analog(s) (e.g., GLP-1 (7-36) such as beinaglutide)), and vice versa such that the user can choose an optimal combination of the delivery doses based on the initial blood glucose level and the blood glucose level in response to the initial administration. For this purpose, the GLP-1 (and/or GLP-1 analog(s) (e.g., GLP-1 (7-36) such as beinaglutide)) and the insulin (and/or insulin analog(s) (e.g., insulin glargine)) may be provided in small doses such that the user can combine two or more of each to achieve an incremented, desired administration dose.

The following examples are provided to better illustrate the claimed invention and the embodiments described herein, and are not to be interpreted as limiting the scope of the invention. To the extent that specific materials are mentioned, it is merely for purposes of illustration and is not intended to limit the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLE 1

### Effects of Insulin Glargine and Beinaglutide on Blood Glucose in db/db Mice

This example demonstrates the effects of the combination of insulin glargine (IGla) and beinaglutide (Bei) on blood glucose in diabetic db/db mice at various doses and ratios.

### Materials

Animals: Male db/db mice (n=42, Lot No.: DB20190926) were purchased from GemPharmatech Co., Ltd. Upon arrival, the mice were housed in an animal facility at FUDAN University, under constant temperature (20-24° C.) and humidity (40-70%). Housing was provided a 12:12-hour light-dark cycle.

Active agents: Insulin glargine was obtained from Sanofi and stored at a temperature of 2-8° C. Beinaglutide having the sequence of His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 1) was obtained from Benemae (beinaglutide injection, 2 mg/mL) and stored at 2-8° C.

The animals were acclimated in the animal facility for 1 week before the study. The mice (n=5) were randomized into different groups by their blood glucose levels under fed condition and body weight on the day before the study. Each group of the mice were fasted for 2 hours before the first dosing. Two hours after fasting, at time point −15 minutes, the blood glucose level of each mouse was measured by glucose meter (ONETOUCH) and recorded and then either the negative control or a dose of insulin glargine (IGla) was subcutaneously injected to a first site in each mouse. Fifteen minutes after the first injection, at time point 0, the blood glucose level of each mouse was measured and recorded again, immediately followed by a second subcutaneous injection of the negative control or a dose of beinaglutide (Bei) to a different site in each mouse. The blood glucose levels of each mouse were measured at various intervals after the second injection, at 15 minutes, 30 minutes, 60 minutes, 120 minutes, 180 minutes, and 240 minutes, respectively.

Two studies were performed with various doses of beinaglutide as detailed in Table 1 and Table 2 below.

TABLE 1
Animal Study 1

| | | | | | | Ratio |
|---|---|---|---|---|---|---|
| | | | | | | (IGla |
| | | | Time Points | | Volume | (U):Bei |
| No. | Group | n | −15 min | 0 | (ml/kg) | (mg)) |
| 1 | Neg. Control | 5 | Vehicle^{a} | Vehicle^{b} | 2 | n/a |
| 2 | IGla_60 U/kg | 5 | IGla | Vehicle^{b} | 2 | n/a |
| 3 | Bei_2 mg/kg | 5 | Vehicle^{a} | Bei | 2 | n/a |
| 4 | IGla_60 U/kg & | 5 | IGla | Bei | 2 | 300:1 |
| | Bei_0.2 mg/kg | | | | | |
| 5 | IGla_60 U/kg & | 5 | IGla | Bei | 2 | 200:1 |
| | Bei_0.3 mg/kg | | | | | |
| 6 | IGla_60 U/kg & | 5 | IGla | Bei | 2 | 100:1 |
| | Bei_0.6 mg/kg | | | | | |
| 7 | IGla_60 U/kg & | 5 | IGla | Bei | 2 | 50:1 |
| | Bei_1.2 mg/kg | | | | | |
| 8 | IGla_60 U/kg & | 5 | IGla | Bei | 2 | 30:1 |
| | Bei_2 mg/kg | | | | | |

Notes:
^{a}Excipient solution for IGla was used as the vehicle control;
^{b}Excipient solution for beinaglutide was used as the vehicle control.

TABLE 2
Animal Study 2

| | | | | | | Ratio |
|---|---|---|---|---|---|---|
| | | | | | | (IGla |
| | | | Time Points | | Volume | (U):Bei |
| No. | Group | n | −15 min | 0 | (ml/kg) | (mg)) |
| 1 | Neg. Control | 5 | Vehicle^{a} | Vehicle^{b} | 2 | n/a |
| 2 | IGla_60 U/kg | 5 | IGla | Vehicle^{b} | 2 | n/a |
| 3 | Bei_0.3 mg/kg | 5 | Vehicle^{a} | Bei | 2 | n/a |
| 4 | IGla_60 U/kg & | 5 | IGla | Bei | 2 | 600:1 |
| | Bei_0.1 mg/kg | | | | | |
| 5 | IGla_60 U/kg & | 5 | IGla | Bei | 2 | 250:1 |
| | Bei_0.24 mg/kg | | | | | |
| 6 | IGla_60 U/kg & | 5 | IGla | Bei | 2 | 200:1 |
| | Bei_0.3 mg/kg | | | | | |
| 7 | IGla_60 U/kg & | 5 | IGla | Bei | 2 | 100:1 |
| | Bei_0.6 mg/kg | | | | | |
| 8 | IGla_60 U/kg & | 5 | IGla | Bei | 2 | 50:1 |
| | Bei_1.2 mg/kg | | | | | |

Notes:
^{a}Excipient solution for IGla was used as the vehicle control;
^{b}Excipient solution for beinaglutide was used as the vehicle control.

FIG. 1 and FIG. 2 show the blood glucose levels of the mice over a period of 4 hours after beinaglutide injection for Study 1 and Study 2, respectively. Subjects treated with higher dose of insulin and/or insulin analogs are more likely to have hypoglycemia that can be problematic and dangerous. No hypoglycemia was observed in the subjects observed in the studies. The insulin glargine dosage was the same (60 U/kg) for all subjects treated with either monotherapy of insulin glargine (FIGS. 1 and 2, hollow square, IGlA_60 U/kg) or a beinaglutide-insulin glargine combinational therapy. The combinational therapy showed a synergistic effect in lowering the blood glucose levels in the subjects treated. For example, the combinational therapy having a beinaglutide dosage of as low as 0.1 mg/kg (FIG. 2, hollow reversed triangle, Bei_0.1 mpk) lowered the blood glucose level more rapidly compared to the subjects treated with insulin glargine only (FIG. 2, hollow square, IGla_60 U/kg) or beinaglutide only (FIG. 2, solid triangle, Bei_0.3 mpk). See also results of combinational therapies having a beinaglutide dosage of 0.24 mg/kg (FIG. 2, solid diamond, Bei_0.24 mpk), a beinaglutide dosage of 0.3 mg/kg (FIG. 2, hollow circle, Bei_0.3 mpk), a beinaglutide dosage of 0.6 mg/kg (FIG. 2, solid square, Bei_0.6 mpk), beinaglutide dosage of 1.2 mg/kg (FIG. 2, hollow triangle, Bei_1.2 mpk). Similarly, the combinational therapy having a beinaglutide dosage of as low as 0.2 mg/kg (FIG. 1, hollow reversed triangle, Bei_0.2 mpk) lowered the blood glucose level more rapidly compared to the subjects treated with insulin glargine only (FIG. 1, hollow square, IGla_60 U/kg) or beinaglutide only (FIG. 1, solid triangle, Bei_2 mpk). See also results of combinational therapies having a beinaglutide dosage of 0.3 mg/kg (FIG. 1, solid diamond, Bei_0.3 mpk), a beinaglutide dosage of 0.6 mg/kg (FIG. 1, hollow circle, Bei_0.6 mpk), a beinaglutide dosage of 1.2 mg/kg (FIG. 1, solid square, Bei_1.2 mpk), a beinaglutide dosage of 2 mg/kg (FIG. 1, hollow triangle, Bei_2 mpk).

The blood sugar level of subjects treated with beinaglutide monotherapy raised back to the same level of the negative control groups (FIG. 1, solid circle, Vehicle) four hour after a beinaglutide treatment of 2 mg/kg (FIG. 1, solid triangle, Bei_2 mpk) or sooner. The blood sugar level of subjects treated with beinaglutide monotherapy raised back to the same level of the negative control groups (FIG. 2, solid circle, Vehicle) 1.5 hour after a beinaglutide treatment of 0.3 mg/kg (FIG. 2, hollow square, Bei_0.3 mpk) or sooner. However, subjects treated with several combinational therapies of beinaglutide and insulin glargine had a blood glucose level lower than that of subjects treated with insulin glargine alone. See, e.g., combinational therapies with a beinaglutide dosage of 0.6 mg/kg (FIG. 1, hollow circle, Bei_0.6 mpk), a beinaglutide dosage of 1.2 mg/kg (FIG. 1, solid square, Bei_1.2 mpk), a beinaglutide dosage of 2 mg/kg (FIG. 1, hollow triangle, Bei_2 mpk) comparing to the subjects treated with insulin glargine only (FIG. 1, hollow square, IGla_60 U/kg); and combinational therapies with a beinaglutide dosage of 0.6 mg/kg (FIG. 2, solid square, Bei_0.6 mpk) and beinaglutide dosage of 1.2 mg/kg (FIG. 2, hollow triangle, Bei_1.2 mpk) comparing to the subjects treated with insulin glargine only (FIG. 2, hollow square, IGla_60 U/kg),

Thus, subjects treated the various combinational therapies of beinaglutide and insulin glargine showed synergistically improved blood glucose control compared to subjects treated with only beinaglutide or insulin glargine.

### EXAMPLE 2

### Effects of Insulin Glargine and Beinaglutide on Blood Glucose in Streptozocin (STZ)-Induced Diabetic C57BL/6 Mice

This example demonstrates the effects of the combination of insulin glargine (IGla) and beinaglutide (Bei) on blood glucose in STZ-induced diabetic C57BL/6 mice at fixed ratio.

Animals: Male C57BL/6 mice (n=70) were purchased from Shanghai Jihui Laboratory Animal Care Co., Ltd. Upon arrival, the mice were housed in an animal facility at Shanghai University of Medicine & Health Sciences, under constant temperature (20-24° C.) and humidity (40-70%). Housing was provided at 12:12-hour light-dark cycle.

Active agents: Insulin glargine was obtained from Sanofi and stored at a temperature of 2-8° C. Beinaglutide having the sequence of His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 1) was obtained from Benemae (beinaglutide injection, 2 mg/mL) and stored at 2-8° C.

After one-week acclimation, the mice were fed with high fat diet with 60 kcal % fat (D12492, Research Diets) for 3 weeks before STZ injection. Diabetic model was induced with 5 consecutive days of multiple low-dose STZ injection (50 mg/kg, intraperitoneally; i.p.). During modeling period, the mice were fasted for 4 hours before STZ treatment each day. The STZ solution (5 mg/ml) was freshly prepared in 0.1 M sodium citrate buffer (pH 4.5) before use and injected within 10 minutes of preparation. Seven days later, the mice with non-fasted blood glucose between 16.7 mM and 27.7 mM were selected and randomized into different groups by blood glucose level and body weight. At 9 am (T=0 minute) of Day 1, the mice were subcutaneously administrated with vehicle, insulin glargine, beinaglutide, or a mixture of insulin glargine and beinaglutide, which were mixed immediately before injection (Table 3). After first injection, tail blood glucose levels were measured at various intervals after injection, at 15 minutes, 30 minutes, 60 minutes, 120 minutes, 180 minutes, and 240 minutes, respectively. Subsequently, the mice of four groups (No. 1, No. 2, No. 4, and No. 6) continued to receive multiple dosing for 4 weeks. Body weight was measured twice per week. On day 28, after blood glucose measurement, the mice were fasted for 6 hours from 9 am, followed by an oral glucose tolerance test (glucose solution: 1 g/kg, 10 ml/kg). The blood glucose was monitored at 0 minute (pre-glucose loading), 15 minutes, 30 minutes, 60 minutes and 120 minutes after glucose loading.

TABLE 3
Animal Study on STZ-Induced Diabetic C57BL/6 Mice

| Group | | | Volume | | |
|---|---|---|---|---|---|
| No. | Treatment | n | (ml/kg) | Route | Frequency |
| 1 | Vehicle | 6 | 3 | s.c. | 1st week: bid; |
| | | | | | 2nd to 4th week: tid |
| 2 | Beinaglutice_0.1 | 6 | 3 | s.c. | 1st week: bid; |
| | mg/kg | | | | 2nd to 4th week: tid |
| 3 | Beinaglutice_2.4 | 6 | 3 | s.c. | Single dose on |
| | mg/kg | | | | Day 1 |
| 4 | Insulin Glargine_5 | 6 | 3 | s.c. | 1st week: bid; |
| | U/kg | | | | 2nd to 4th week: tid |
| 5 | Insulin Glargine_10 | 6 | 3 | s.c. | Single dose on |
| | U/kg | | | | Day 1 |
| 6 | IGlar_5 U/kg and | 6 | 3 | s.c. | 1st week: bid; |
| | Bei_0.05 mg/kg | | | | 2nd to 4th week: tid |
| 7 | IGlar_10 U/kg and | 6 | 3 | s.c. | Single dose on |
| | Bei_0.1 mg/kg | | | | Day 1 |

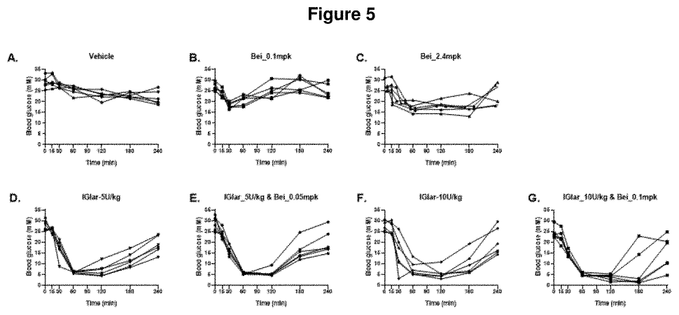

Blood glucose after single dosing: After a single administration, beinaglutide (Bei) and insulin glargine (IGlar) significantly reduced blood glucose in STZ-induced hyperglycemic mice. Based on the area under curve (AUC) of blood glucose, significant reduction of blood glucose was observed in Bei_2.4 mg/kg, IGlar_5 U/kg, IGlar_10 U/kg, and the two combo groups. Although no significant difference between IGlar and its combo group was observed, coefficient of variance (CV) of the combo group was smaller compared to IGlar alone at some time points. At 30 minutes, the CV value was 0.2573 for the IGlar_5 U/kg group and 0.1475 for the IGlar_5 U/kg and Bei_0.05 mg/kg combo group, respectively. At 120 minutes, the CV value was 0.4151 for the IGlar_5 U/kg group and 0.3102 for the IGlar_5 U/kg and Bei_0.05 mg/kg combo group, respectively. The CV value was 0.5517 at 30 minutes and 0.4240 at 60 minutes for the IGlar_10 U/kg group, whereas the CV was 0.1023 at 30 minutes and 0.1319 at 60 minutes for the IGlar_10 U/kg and Bei_0.1 mg/kg combo group. The minimum blood glucose of the IGlar_5 U/kg group and the IGlar_5 U/kg and Bei_0.05 mg/kg combo group was 6.07±0.43 mM and 5.67±0.47 mM at 60 minutes, respectively. For the IGlar_10 U/kg group and IGlar_10 U/kg and Bei_0.1 mg/kg combo group, the minimum blood glucose was 5.50±2.74 mM and 3.75±1.37 mM at 120 minutes, respectively. At 180 minutes, the minimum blood glucose was 9.95±5.22 mM for the IGlar_10 U/kg group and 7.50±9.04 mM for the IGlar_10 U/kg and Bei_0.1 mg/kg combo group, respectively. Collectively, the efficacy of the combo group in glycemic control was more stable, durable and faster compared to IGlar administration alone. The results are shown in FIGS. 3-5 and summarized in Table 4 below.

TABLE 4
Test Results of Various Treatment Groups (Single Dose)

| | | Animal | 0 | 15 | 30 | 60 | 120 | 180 | 240 | AUC(0- |
|---|---|---|---|---|---|---|---|---|---|---|
| Group | Treatment | No. | min | min | min | min | min | min | min | 120 min) |
| 1 | Vehicle | 13 | 25.4 | 25.9 | 26.6 | 21.7 | 22.9 | 24.7 | 19.1 | 5583 |
| 1 | Vehicle | 18 | 28.3 | 29 | 27.8 | 25.7 | 25.7 | 22.2 | 21.3 | 5942 |
| 1 | Vehicle | 33 | 27.7 | 28.4 | 28.8 | 26.2 | 19.6 | 24.2 | 24.6 | 5827 |
| 1 | Vehicle | 37 | 29.8 | 27.9 | 26.2 | 24.6 | 22.2 | 22.1 | 19.9 | 5594 |
| 1 | Vehicle | 57 | 30.3 | 32.8 | 26.4 | 26.6 | 23.7 | 21.4 | 18.6 | 5774 |
| 1 | Vehicle | 60 | 33.2 | 33.3 | 28.2 | 27.3 | 23.3 | 22.8 | 26.7 | 6179 |
| MEAN | | | 29.12 | 29.55 | 27.33 | 25.35 | 22.90 | 22.90 | 21.70 | 5816 |
| SD | | | 2.65 | 2.91 | 1.08 | 2.00 | 2.00 | 1.29 | 3.26 | 224.94 |
| CV | | | 0.0909 | 0.0984 | 0.0394 | 0.0791 | 0.0874 | 0.0563 | 0.1503 | 0.0387 |
| 2 | Beinaglutide_0.1 mpk | 6 | 25.9 | 24.8 | 16.3 | 21.5 | 21.8 | 25.3 | 22.1 | 5390 |
| 2 | Beinaglutide_0.1 mpk | 12 | 24.8 | 22.4 | 17.7 | 18.4 | 24.8 | 30.6 | 23.8 | 5786 |
| 2 | Beinaglutide_0.1 mpk | 15 | 25.3 | 21.7 | 20.2 | 23.2 | 21.3 | 32 | 22.9 | 5899 |
| 2 | Beinaglutide_0.1 mpk | 20 | 26.2 | 24.6 | 17.4 | 17.6 | 24.1 | 24.2 | 21.8 | 5301 |
| 2 | Beinaglutide_0.1 mpk | 27 | 28.2 | 22.6 | 19.3 | 21.8 | 30.7 | 30.3 | 28.2 | 6472 |
| 2 | Beinaglutide_0.1 mpk | 34 | 29.6 | 26.8 | 18.7 | 21.5 | 26.3 | 25.4 | 29.9 | 6011 |
| MEAN | | | 26.67 | 23.82 | 18.27 | 20.67 | 24.83 | 27.97 | 24.78 | 5390 |
| SD | | | 1.85 | 1.92 | 1.41 | 2.17 | 3.43 | 3.36 | 3.42 | 429.59 |
| CV | | | 0.0694 | 0.0807 | 0.0772 | 0.1052 | 0.1381 | 0.1202 | 0.1380 | 0.0739 |
| P (vs. group1) | | | 0.0928 | 0.0024 | 0.0000 | 0.0031 | 0.2605 | 0.0063 | 0.1411 | 0.9740 |
| 3 | Beinaglutide_2.4 mpk | 10 | 23.9 | 25.7 | 19.63 | 17.1 | 18.8 | 17.3 | 27.4 | 4764 |
| 3 | Beinaglutide_2.4 mpk | 17 | 24.9 | 27.2 | 19.8 | 15.9 | 18 | 17.9 | 28.9 | 4777 |
| 3 | Beinaglutide_2.4 mpk | 22 | 26.4 | 27.8 | 18.6 | 14.4 | 14.4 | 13.2 | 27.6 | 4166 |
| 3 | Beinaglutide_2.4 mpk | 32 | 31.1 | 31.7 | 20.8 | 16.6 | 16.3 | 16.7 | 18.1 | 4447 |
| 3 | Beinaglutide_2.4 mpk | 41 | 26.8 | 26.8 | 19.3 | 17.8 | 21.5 | 23.8 | 20.1 | 5159 |
| 3 | Beinaglutide_2.4 mpk | 51 | 25.1 | 24.4 | 20.3 | 20.7 | 17.8 | 16.6 | 18.6 | 4565 |
| MEAN | | | 26.37 | 27.27 | 19.74 | 17.08 | 17.80 | 17.58 | 23.45 | 4764 |
| SD | | | 2.55 | 2.48 | 0.77 | 2.12 | 2.39 | 3.46 | 5.02 | 338.13 |
| CV | | | 0.0966 | 0.0911 | 0.0389 | 0.1240 | 0.1342 | 0.1967 | 0.2140 | 0.0728 |
| P (vs. group1) | | | 0.0966 | 0.1743 | 0.0000 | 0.0000 | 0.0025 | 0.0055 | 0.4902 | 0.0000 |
| 4 | Insulin glargine_5 U/kg | 3 | 31.1 | 23.4 | 19.7 | 6.4 | 5.6 | 10.8 | 18.8 | 2864 |
| 4 | Insulin glargine_5 U/kg | 5 | 29.3 | 25.7 | 8.7 | 6.2 | 7.5 | 14.1 | 23.2 | 3072 |
| 4 | Insulin glargine_5 U/kg | 8 | 25.9 | 26.6 | 17.9 | 5.9 | 12.2 | 17.2 | 23.5 | 3731 |
| 4 | Insulin glargine_5 U/kg | 39 | 28.3 | 24.1 | 16.6 | 5.4 | 4.4 | 8.9 | 16.7 | 2489 |
| 4 | Insulin glargine_5 U/kg | 53 | 25.3 | 26.3 | 21.2 | 6.6 | 7.8 | 11.5 | 17.7 | 3047 |
| | Insulin glargine_5 U/kg | 63 | 25.4 | 26 | 17.6 | 5.9 | 4.6 | 8.3 | 13.1 | 2409 |
| MEAN | | | 27.55 | 25.35 | 16.95 | 6.07 | 7.02 | 11.80 | 18.83 | 2864 |
| SD | | | 2.39 | 1.29 | 4.36 | 0.43 | 2.91 | 3.35 | 3.99 | 478.47 |
| CV | | | 0.0869 | 0.0511 | 0.2573 | 0.0704 | 0.4151 | 0.2841 | 0.2118 | 0.1630 |
| P (vs. group1) | | | 0.3074 | 0.0090 | 0.0002 | 0.0000 | 0.0000 | 0.0000 | 0.2028 | 0.0000 |
| 5 | Insulin glargine_10 U/kg | 19 | 26.7 | 24.4 | 17.3 | 9.6 | 10.8 | 19.3 | 26.4 | 3986 |
| 5 | Insulin glargine_10 U/kg | 26 | 25.5 | 23.6 | 10.6 | 5 | 4.2 | 9.4 | 16.1 | 2308 |
| 5 | Insulin glargine_10 U/kg | 31 | 29 | 30.3 | 3.1 | 7 | 5.4 | 6.1 | 15.4 | 2209 |
| 5 | Insulin glargine_10 U/kg | 47 | 24.6 | 24.4 | 11.1 | 5.2 | 3 | 5.8 | 14.3 | 1991 |
| 5 | Insulin glargine_10 U/kg | 49 | 30.2 | 29 | 26.1 | 13.3 | 5.3 | 6.7 | 19.3 | 3146 |
| 5 | Insulin glargine_10 U/kg | 56 | 30.6 | 28.4 | 19.9 | 5.8 | 4.3 | 12.4 | 29.6 | 3254 |
| MEAN | | | 27.77 | 26.68 | 14.68 | 7.65 | 5.50 | 9.95 | 20.18 | 3986 |
| SD | | | 2.52 | 2.88 | 8.10 | 3.24 | 2.74 | 5.22 | 6.36 | 771.51 |
| CV | | | 0.0908 | 0.1077 | 0.5517 | 0.4240 | 0.4981 | 0.5248 | 0.3151 | 0.2740 |
| P (vs. group1) | | | 0.3869 | 0.1167 | 0.0035 | 0.0000 | 0.0000 | 0.0002 | 0.6145 | 0.0000 |
| 6 | IGlar_5 U/kg&Bei_0.05 mpk | 1 | 32.8 | 21.4 | 15.8 | 5.9 | 4.6 | 12 | 14.8 | 2628 |
| 6 | IGlar_5 U/kg&Bei_0.05 mpk | 4 | 24.7 | 23.9 | 19.3 | 6.2 | 5.2 | 13.9 | 17.8 | 2937 |
| 6 | IGlar_5 U/kg&Bei_0.05 mpk | 23 | 30.5 | 24.9 | 13.3 | 5.2 | 9.4 | 24.6 | 29.4 | 4058 |
| 6 | IGlar_5 U/kg&Bei_0.05 mpk | 28 | 28 | 22.3 | 14.7 | 6.1 | 5.2 | 15.7 | 17.2 | 2920 |
| 6 | IGlar_5 U/kg&Bei_0.05 mpk | 30 | 31.1 | 27.9 | 19.3 | 5.5 | 5.4 | 16.7 | 23.8 | 3374 |
| 6 | IGlar_5 U/kg&Bei_0.05 mpk | 48 | 25.8 | 22.9 | 16.9 | 5.1 | 4.9 | 13.4 | 16.8 | 2749 |
| MEAN | | | 28.82 | 23.88 | 16.55 | 5.67 | 5.78 | 16.05 | 19.97 | 2628 |
| SD | | | 3.18 | 2.32 | 2.44 | 0.47 | 1.79 | 4.51 | 5.53 | 528.42 |
| CV | | | 0.1104 | 0.0970 | 0.1475 | 0.0825 | 0.3102 | 0.2810 | 0.2768 | 0.1699 |
| P (vs. group1) | | | 0.8626 | 0.0039 | 0.0000 | 0.0000 | 0.0000 | 0.0050 | 0.5232 | 0.0000 |
| P (vs. group4) | | | 0.4539 | 0.2054 | 0.8485 | 0.1530 | 0.3978 | 0.0937 | 0.6924 | 0.5599 |
| 7 | IGlar_10 U/kg&Bei_0.1 mpk | 21 | 22.4 | 18.3 | 13.6 | 4.6 | 4.3 | 14.3 | 24.8 | 2816 |
| 7 | IGlar_10 U/kg&Bei_0.1 mpk | 36 | 22.5 | 21.4 | 15.2 | 4.7 | 4.7 | 3.1 | 19.7 | 2102 |
| 7 | IGlar_10 U/kg&Bei_0.1 mpk | 44 | 23.1 | 21.6 | 13.6 | 4.8 | 3.9 | 1.4 | 10.3 | 1646 |
| 7 | IGlar_10 U/kg&Bei_0.1 mpk | 58 | 24.2 | 21.8 | 14.1 | 4.7 | 1.6 | 2.1 | 10.5 | 1574 |
| 7 | IGlar_10 U/kg&Bei_0.1 mpk | 61 | 23.8 | 24.1 | 13.3 | 5.9 | 2.7 | 1.2 | 4.7 | 1480 |
| 7 | IGlar_10 U/kg&Bei_0.1 mpk | 65 | 29.4 | 27.4 | 17.2 | 6.1 | 5.3 | 22.9 | 20.3 | 3594 |

| | | | | | | | | |
|---|---|---|---|---|---|---|---|---|
| MEAN | 24.23 | 22.43 | 14.50 | 5.13 | 3.75 | 7.50 | 15.05 | 2816 |
| SD | 2.63 | 3.06 | 1.48 | 0.68 | 1.37 | 9.04 | 7.68 | 842.45 |
| CV | 0.1084 | 0.1362 | 0.1023 | 0.1319 | 0.3648 | 1.2056 | 0.5101 | 0.3826 |
| P (vs. group1) | 0.0094 | 0.0020 | 0.0000 | 0.0000 | 0.0000 | 0.0020 | 0.0793 | 0.0000 |
| P (vs. group5) | 0.0388 | 0.0325 | 0.9576 | 0.0924 | 0.1918 | 0.5782 | 0.2358 | 0.2176 |

Blood glucose levels and body weight changes after multiple dosing: As shown in FIGS. 6 and 7, during the 4-week treatment, IGlar alone induced weight gain of 2.4±0.4 g on Day 28, in comparison to 1.4±0.3 g of the vehicle group. In contrast, beinaglutide reduced body weight with 1.0±0.3 g decrease on Day 28. For the combo group, weight gain on Day 28 was 1.3±0.3 g, which was significantly lower than the weight gain of the IGlar_5 U/kg group. The results are summarized in Table 5 below.

TABLE 5
Test Results of Various Treatment Groups (Multiple Doses)

| | Animal | Day | Day | Day | Day | Day | Day | Day | Day | Day | Day | Day | Day | Day | Day |
|---|---|---|---|---|---|---|---|---|---|---|---|---|---|---|---|
| Treatment | No. | 0 | 4 | 7 | 8 | 10 | 12 | 14 | 16 | 18 | 20 | 22 | 24 | 26 | 28 |
| Vehicle | 13 | 0.0 | 0.4 | 0.9 | 0.5 | 0.7 | 0.2 | 0.9 | 1.3 | 1.1 | 0.6 | 0.8 | 1.3 | 1.3 | 1.6 |
| Vehicle | 18 | 0.0 | −0.1 | 0.3 | −0.3 | −0.2 | −0.6 | 0.0 | 0.1 | 0.0 | −0.4 | −0.4 | −0.5 | 0.7 | 0.3 |
| Vehicle | 33 | 0.0 | −0.1 | 0.0 | −0.3 | 0.2 | −0.6 | 0.4 | 0.6 | 0.6 | 0.8 | 0.8 | 1.1 | 1.5 | 1.5 |
| Vehicle | 37 | 0.0 | 0.2 | 0.7 | 0.4 | 0.4 | 0.6 | 0.5 | 0.2 | 0.6 | 0.3 | 0.5 | 0.3 | 0.6 | 1.1 |
| Vehicle | 57 | 0.0 | 0.5 | 0.8 | 0.6 | 0.4 | 1.0 | 1.7 | 2.2 | 1.9 | 3.0 | 2.6 | 2.7 | 3.5 | 3.0 |
| Vehicle | 60 | 0.0 | −0.4 | 0.1 | 0.5 | 0.0 | 0.6 | 0.4 | 0.5 | 0.9 | 0.8 | 0.4 | 0.3 | 0.7 | 1.1 |
| MEAN | | 0.0 | 0.1 | 0.5 | 0.2 | 0.3 | 0.2 | 0.7 | 0.8 | 0.9 | 0.9 | 0.8 | 0.9 | 1.4 | 1.4 |
| SEM | | 0.0 | 0.1 | 0.1 | 0.2 | 0.1 | 0.2 | 0.2 | 0.3 | 0.2 | 0.4 | 0.4 | 0.4 | 0.4 | 0.3 |
| Beinaglutide_0.1 mpk | 6 | 0.0 | −1.2 | −0.8 | −1.4 | −1.7 | −1.9 | −1.3 | −1.3 | −2.3 | −0.9 | −1.1 | −0.9 | −0.8 | −0.4 |
| Beinaglutide_0.1 mpk | 12 | 0.0 | −0.6 | −0.6 | −1.1 | −1.5 | −1.6 | −1.5 | −1.6 | −1.9 | −2.0 | −2.1 | −1.5 | −1.7 | −1.6 |
| Beinaglutide_0.1 mpk | 15 | 0.0 | −0.3 | −0.8 | −0.9 | −0.6 | −1.2 | −0.5 | −0.4 | −0.6 | −0.3 | −0.2 | 0.0 | −0.1 | 0.0 |
| Beinaglutide_0.1 mpk | 20 | 0.0 | −0.2 | −0.8 | −0.9 | −0.8 | −1.0 | −1.2 | −1.1 | −1.5 | −1.6 | −0.9 | −1.0 | −1.6 | −1.1 |
| Beinaglutide_0.1 mpk | 27 | 0.0 | −0.1 | −0.7 | −1.2 | −0.8 | −0.6 | −1.0 | −1.4 | −0.9 | −1.6 | −1.1 | −1.5 | −2.3 | −2.1 |
| Beinaglutide_0.1 mpk | 34 | 0.0 | −1.1 | −1.2 | −1.4 | −1.0 | −0.5 | −0.8 | −1.4 | −1.2 | −0.9 | −1.7 | −1.5 | −1.3 | −0.9 |
| MEAN | | 0.0 | −0.6 | −0.8 | −1.1 | −1.1 | −1.1 | −1.0 | −1.2 | −1.4 | −1.2 | −1.2 | −1.1 | −1.3 | −1.0 |
| SEM | | 0.0 | 0.2 | 0.1 | 0.1 | 0.2 | 0.2 | 0.1 | 0.2 | 0.2 | 0.2 | 0.2 | 0.2 | 0.3 | 0.3 |
| IGlar_5 U/kg | 3 | 0.0 | −0.2 | 0.2 | 0.0 | 0.1 | 0.2 | 0.3 | 0.7 | 0.9 | 1.0 | 1.2 | 1.3 | 1.5 | 1.7 |
| IGlar_5 U/kg | 5 | 0.0 | 0.0 | 0.7 | 1.1 | 1.0 | 0.9 | 1.3 | Null | Null | Null | Null | Null | Null | Null |
| IGlar_5 U/kg | 8 | 0.0 | 0.6 | 1.0 | 0.6 | 0.7 | 0.4 | 0.8 | 1.3 | 1.2 | 1.9 | 1.7 | 1.9 | 2.2 | 2.3 |
| IGlar_5 U/kg | 39 | 0.0 | −0.1 | 1.0 | 0.7 | 1.0 | 0.8 | 2.0 | 1.7 | 2.4 | 2.3 | 2.1 | 2.3 | 3.3 | 2.9 |
| IGlar_5 U/kg | 53 | 0.0 | 0.3 | 0.8 | 1.3 | 1.1 | 1.3 | 1.9 | 1.7 | 2.4 | 2.5 | 2.6 | 2.7 | 3.7 | 3.8 |
| IGlar_5 U/kg | 63 | 0.0 | −0.4 | 0.5 | 0.4 | 0.8 | 0.6 | 0.8 | 1.0 | 1.3 | 1.0 | 1.0 | 0.6 | 1.2 | 1.4 |
| MEAN | | 0.0 | 0.0 | 0.7 | 0.7 | 0.8 | 0.7 | 1.2 | 1.3 | 1.6 | 1.7 | 1.7 | 1.8 | 2.4 | 2.4 |
| SEM | | 0.0 | 0.1 | 0.1 | 0.2 | 0.1 | 0.1 | 0.3 | 0.2 | 0.3 | 0.3 | 0.3 | 0.3 | 0.4 | 0.4 |
| IGlar_5 U/kg& | 1 | 0.0 | 0.3 | 1.0 | 0.7 | 0.3 | 0.7 | 0.4 | 1.0 | 1.3 | 1.4 | 1.1 | 1.0 | 1.5 | 1.2 |
| Bei_0.05 mpk | | | | | | | | | | | | | | | |
| IGlar_5 U/kg& | 4 | 0.0 | −0.6 | −0.4 | −0.1 | −0.2 | −0.1 | −0.2 | 0.1 | 0.2 | −0.1 | 0.2 | 0.2 | 0.3 | 0.1 |
| Bei_0.05 mpk | | | | | | | | | | | | | | | |
| IGlar_5 U/kg& | 23 | 0.0 | 0.4 | 1.0 | 1.3 | 1.4 | 1.3 | 1.1 | 1.8 | 2.1 | 1.2 | 1.6 | 1.7 | 2.0 | 1.6 |
| Bei_0.05 mpk | | | | | | | | | | | | | | | |
| IGlar_5 U/kg& | 28 | 0.0 | 0.7 | 0.6 | 0.2 | 1.3 | 1.1 | 1.1 | 1.0 | 1.0 | 1.3 | 1.4 | 1.9 | 2.0 | 1.9 |
| Bei_0.05 mpk | | | | | | | | | | | | | | | |
| IGlar_5 U/kg& | 30 | 0.0 | 0.4 | 0.0 | −0.1 | 0.5 | 0.8 | 0.4 | 0.4 | 0.7 | 0.6 | 0.3 | 0.5 | 1.8 | 1.9 |
| Bei_0.05 mpk | | | | | | | | | | | | | | | |
| IGlar_5 U/kg& | 48 | 0.0 | 0.3 | 0.2 | 0.2 | 0.0 | 0.6 | 0.7 | 0.3 | 0.8 | 0.3 | 0.7 | 0.3 | 1.0 | 1.0 |
| Bei_0.05 mpk | | | | | | | | | | | | | | | |

| | | | | | | | | | | | | | | |
|---|---|---|---|---|---|---|---|---|---|---|---|---|---|---|
| MEAN | 0.0 | 0.2 | 0.4 | 0.4 | 0.5 | 0.7 | 0.6 | 0.8 | 1.0 | 0.8 | 0.9 | 0.9 | 1.4 | 1.3 |
| SEM | 0.0 | 0.2 | 0.2 | 0.2 | 0.2 | 0.2 | 0.2 | 0.2 | 0.2 | 0.2 | 0.2 | 0.3 | 0.3 | 0.3 |

Null means that the animal was dead.

On Day 28, the fasting blood glucose was measured after 6 hours fasting. The data indicated that IGlar_5 U/kg and combo group significantly reduced fasting blood glucose at 14.0±2.43 mM and 11.7±0.92 mM, respectively, compared to vehicle group of 20.5±1.50 mM. The results are shown in FIG. 8 and Table 6 below.

TABLE 6
Test Results of Various Treatment Groups (Fasting)

| Treatment | No. 1 | No. 2 | No. 3 | No. 4 | No. 5 | No. 6 | Mean | SEM |
|---|---|---|---|---|---|---|---|---|
| Vehicle | 21.8 | 16.8 | 20.4 | 26.6 | 16.7 | 20.8 | 20.5 | 1.50 |
| Bei_0.1 mpk | 18.5 | 23.6 | 18.2 | 17.8 | 19 | 11.6 | 18.1 | 1.57 |
| IGlar_5 U/kg | 16.1 | Null | 9.4 | 7.6 | 15.9 | 20.9 | 14.0 | 2.43 |
| IGlar_5 U/kg & Bei_0.05 mpk | 14.2 | 12.2 | 8.7 | 9.4 | 11.7 | 13.8 | 11.7 | 0.92 |

Null means that the animal was dead.

During oral glucose tolerance test (OGTT) on Day 28, all test groups including beinaglutide alone, insulin glargine alone, and the combo group significantly reduced blood glucose based on the AUC. Additionally, the combo group had a synergistic effect in lowering the blood glucose level compared to beinaglutide alone and insulin glargine alone. The results are shown in FIGS. 9 and 10 and Table 7 below.

TABLE 7
OGTT Test Results of Various Treatment Groups

| | Animal | 0 | 15 | 30 | 60 | 120 | |
|---|---|---|---|---|---|---|---|
| Treatment | No. | min | min | min | min | min | AUC |
| Vehicle | 13 | 21.8 | 33.3 | 32.4 | 26.7 | 22.8 | 3277.5 |
| Vehicle | 18 | 16.8 | 29.8 | 23.2 | 20.7 | 17.3 | 2545.5 |
| Vehicle | 33 | 20.4 | 31.4 | 29.8 | 24.6 | 22.2 | 3067.5 |
| Vehicle | 37 | 26.6 | 33.3 | 33.3 | 33.3 | 23.9 | 3663.8 |
| Vehicle | 57 | 16.7 | 28.8 | 33.3 | 31.8 | 28.2 | 3583.5 |
| Vehicle | 60 | 20.8 | 33.3 | 33.1 | 23.3 | 21.6 | 3096.8 |
| MEAN | | 20.52 | 31.65 | 30.85 | 26.73 | 22.67 | 3205.75 |
| SEM | | 1.50 | 0.81 | 1.62 | 2.01 | 1.44 | 165.78 |
| Beinaglutide_0.1 | 6 | 18.5 | 31.4 | 25.5 | 21 | 17.3 | 2647.5 |
| mpk | | | | | | | |
| Beinaglutide_0.1 | 12 | 23.6 | 31.6 | 28 | 25.6 | 18.6 | 2991.0 |
| mpk | | | | | | | |
| Beinaglutide_0.1 | 15 | 18.2 | 28.1 | 26.8 | 19.4 | 20.5 | 2649.0 |
| mpk | | | | | | | |
| Beinaglutide_0.1 | 20 | 17.8 | 28.4 | 23.4 | 18.8 | 16.6 | 2430.0 |
| mpk | | | | | | | |
| Beinaglutide_0.1 | 27 | 19 | 29.8 | 27.7 | 23.1 | 20.1 | 2855.3 |
| mpk | | | | | | | |
| Beinaglutide_0.1 | 34 | 11.6 | 25.9 | 25.8 | 19.4 | 15.4 | 2391.0 |
| mpk | | | | | | | |
| MEAN | | 18.12 | 29.20 | 26.20 | 21.22 | 18.08 | 2660.63 |
| SEM | | 1.57 | 0.89 | 0.69 | 1.08 | 0.82 | 95.43 |
| IGlar_5 U/kg | 3 | 16.1 | 33.3 | 27.9 | 23 | 17.9 | 2820.0 |
| IGlar_5 U/kg | 5 | Null | Null | Null | Null | Null | Null |
| IGlar_5 U/kg | 8 | 9.4 | 25.4 | 23.4 | 18.6 | 13 | 2205.0 |
| IGlar_5 U/kg | 39 | 7.6 | 24 | 23.1 | 19 | 12.9 | 2178.8 |
| IGlar_5 U/kg | 53 | 15.9 | 25.9 | 23.3 | 16.4 | 12.1 | 2133.0 |
| IGlar_5 U/kg | 63 | 20.9 | 30.9 | 25.3 | 23.2 | 12.7 | 2614.5 |
| MEAN | | 13.98 | 27.90 | 24.60 | 20.04 | 13.72 | 2390.25 |
| SEM | | 2.43 | 1.78 | 0.92 | 1.33 | 1.06 | 137.88 |
| IGlar_5 U/kg& | 1 | 14.2 | 26.1 | 22.5 | 19.9 | 13.4 | 2301.8 |
| Bei_0.05 mpk | | | | | | | |
| IGlar_5 U/kg& | 4 | 12.2 | 26 | 23.2 | 17.9 | 13.3 | 2208.0 |
| Bei_0.05 mpk | | | | | | | |
| IGlar_5 U/kg& | 23 | 8.7 | 22.7 | 22.1 | 15.6 | 10.8 | 1929.0 |
| Bei_0.05 mpk | | | | | | | |
| IGlar_5 U/kg& | 28 | 9.4 | 20.9 | 18.9 | 13.1 | 7.7 | 1629.8 |
| Bei_0.05 mpk | | | | | | | |
| IGlar_5 U/kg& | 30 | 11.7 | 25.2 | 24.9 | 19.3 | 11.8 | 2248.5 |
| Bei_0.05 mpk | | | | | | | |
| IGlar_5 U/kg& | 48 | 13.8 | 25.5 | 23.1 | 18.4 | 12.9 | 2220.8 |
| Bei_0.05 mpk | | | | | | | |

| | | | | | | |
|---|---|---|---|---|---|---|
| MEAN | 11.67 | 24.40 | 22.45 | 17.37 | 11.65 | 2089.63 |
| SEM | 0.92 | 0.86 | 0.81 | 1.05 | 0.89 | 106.26 |

1. A method of treating a condition associated with elevated blood glucose in a subject comprising administering a first effective amount of one or more first active ingredients selected from the group consisting of GLP-1 and GLP-1 analogs and a second effective amount of one or more second active ingredients selected from the group consisting of insulin and insulin analogs to a subject to treat the condition.

2. The method of claim 1, wherein the subject has diabetes such as type 1 and type 2 diabetes or has an elevated risk of having elevated blood glucose and/or diabetes such as type 1 and type 2 diabetes.

3. The method of claim 1, wherein the GLP-1 analogs are selected from the group consisting of GLP-1 (7-37), GLP-1 (7-36), and GLP-1 (7-35).

4. The method of claim 1, wherein the second effective amount (U) of the insulin and/or insulin analog(s) and the first effective amount (mg) of the GLP-1 and/or GLP-1 analog(s) are administered in a ratio of between 10:1 and 800:1, about 30:1 to about 500:1m about 50:1 to about 250:1, about 70:1 to about 220:1, or about 100:1 to about 200:1, about 10:1, about 20:1, about 30:1, about 40:1, about 50:1, about 60:1, about 60:1, about 70:1, about 80:1, about 90:1, about 100:1, about 150:1, about 200:1, about 250:1, about 300:1, about 350:1, about 400:1, about 450:1, about 500:1, about 550:1, about 600:1, about 650:1, about 700:1, about 750:1, or about 800:1.

5. The method of claim 1, wherein the GLP-1 and/or GLP-1 analog(s) are administered in a range from about 0.00070 mg/kg to about 0.0197 mg/kg body weight, and/or the insulin and/or insulin analog(s) are administered in a range from about 2 U/d to about 100 U/d, about 6 U/d to about 60 U/d, about 12 U/d to about 40 U/d, about 20 U/d to about 30 U/d, or about 15 U/d.

6. The method of claim 1, wherein the first effective amount of the GLP-1 and/or GLP-1 analog(s) and/or the second effective amount of the insulin and/or insulin analog(s) are administered once a day, twice a day, three times a day, or four times a day.

7. The method of claim 1, wherein the first effective amount of the GLP-1 and/or GLP-1 analog(s) is administered before, during, or after the administration of the second effective amount of the insulin and/or insulin analog(s).

8. The method of claim 1, wherein the GLP-1 and/or GLP-1 analog(s) are administered at a daily dosage of about 0.01 mg/d to about 1 mg/d, about 0.06 mg/d to 0.6 mg/d, or about 0.12 mg/d to about 0.4 mg/d.

9. The method of claim 1, wherein the GLP-1 analog is beinaglutide.

10. A combinational therapy comprising a first effective amount of one or more first active ingredients selected from the group consisting of GLP-1 and GLP-1 analogs and a second effective amount of one or more second active ingredients selected from the group consisting of insulin and insulin analogs for treating a condition associated with elevated blood glucose in a subject.

11. The combinational therapy of claim 10, wherein the subject has diabetes such as type 1 and type 2 diabetes or has an elevated risk of having elevated blood glucose and/or diabetes such as type 1 and type 2 diabetes.

12. The combinational therapy of claim 10, wherein the GLP-1 analog is beinaglutide.

13. The combinational therapy of claim 10, comprising the second effective amount (U) of the insulin and/or insulin analog(s) and the first effective amount (mg) of the GLP-1 and/or GLP-1 analog(s) at a ratio of between 10:1 and 800:1, about 30:1 to about 500:1m about 50:1 to about 250:1, about 70:1 to about 220:1, or about 100:1 to about 200:1, about 10:1, about 20:1, about 30:1, about 40:1, about 50:1, about 60:1, about 60:1, about 70:1, about 80:1, about 90:1, about 100:1, about 150:1, about 200:1, about 250:1, about 300:1, about 350:1, about 400:1, about 450:1, about 500:1, about 550:1, about 600:1, about 650:1, about 700:1, about 750:1, or about 800:1.

14. The combinational therapy of claim 10, wherein the GLP-1 and/or GLP-1 analog(s) are administered in a range from 0.00070 mg/kg to about 0.0197 mg/kg body weight, or the insulin and/or insulin analog(s) are administered in a range from about 2 U/d to about 100 U/d, about 6 U/d to about 60 U/d, about 12 U/d to about 40 U/d, about 20 U/d to about 30 U/d, or about 15 U/d.

15. The combinational therapy of claim 10, wherein 1) the GLP-1 and/or GLP-1 analog(s) and 2) the insulin and/or insulin analog(s) are formulated in a single composition.

16. The combinational therapy of claim 10, wherein 1) the GLP-1 and/or GLP-1 analog(s) and 2) the insulin and/or insulin analog(s) are formulated in two separate compositions.

17. A kit comprising a first effective amount of one or more first active ingredients selected from the group consisting of GLP-1 and GLP-1 analogs and a second effective amount of one or more second active ingredients selected from the group consisting of insulin and insulin analogs for treating a condition associated with elevated blood glucose in a subject.

18. The kit of claim 17, wherein the subject has diabetes such as type 1 and type 2 diabetes or has an elevated risk of having elevated blood glucose and/or diabetes such as type 1 and type 2 diabetes.

19. The kit of claim 17, wherein the GLP-1 analog is beinaglutide.

20. The kit of claim 17, wherein 1) the GLP-1 and/or GLP-1 analog(s) and 2) the insulin and/or insulin analog(s) are formulated in a single composition.

21. (canceled)

22. (canceled)
